(19) United States
(12) Patent Application Publication
(10) Pub. No.: US 2025/0099011 A1
(43) Pub. Date: Mar. 27, 2025

(54) PHYSIOLOGICAL INFORMATION MEASUREMENT SYSTEM AND PHYSIOLOGICAL INFORMATION MEASUREMENT METHOD

(71) Applicant: NIHON KOHDEN CORPORATION, Tokyo (JP)

(72) Inventors: Kei KIMURA, Tokorozawa-shi (JP); Fumiya WADA, Tokorozawa-shi (JP)

(73) Assignee: NIHON KOHDEN CORPORATION, Tokyo (JP)

(21) Appl. No.: 18/890,574

(22) Filed: Sep. 19, 2024

(30) Foreign Application Priority Data
Sep. 27, 2023 (JP) 2023-164677

(51) Int. Cl. A61B 5/366; A61B 5/00; A61B 5/021; A61B 5/0245; A61B 5/352

(52) U.S. Cl. CPC A61B 5/366; A61B 5/0245; A61B 5/352; A61B 5/02108; A61B 5/746

(57) ABSTRACT

The physiological information measurement system includes: a detector configured to detect a candidate QRS complex from an electrocardiogram of a subject; and an estimator configured to estimate whether the candidate QRS complex is a QRS complex based on the candidate QRS complex and information on a cardiac function of the subject.

### CROSS-REFERENCE TO RELATED APPLICATIONS

This application is based on and claims priority under 35 USC 119 from Japanese Patent Application No. 2023-164677 filed on Sep. 27, 2023, the contents of which are incorporated herein by reference.

### TECHNICAL FIELD

The presently disclosed subject matter relates to a physiological information measurement system and a physiological information measurement method.

### BACKGROUND ART

A physiological information measurement system such as a patient monitor measures a heart rate to grasp a state of a patient, and notifies a medical worker by emitting an alarm sound when the heart rate is out of a normal range.

Generally, the heart rate is the number of QRS complexes (heartbeats) generated in one minute. To measure the heart rate in time shorter than one minute, the number of QRS complexes measured in six seconds in an electrocardiogram is multiplied by ten, and the heart rate is calculated based on an RR interval that is an interval of the QRS complexes. For this reason, it is necessary to accurately detect the QRS complexes in the measurement of the heart rate. When trying to detect atrial fibrillation in which generation of the QRS complexes is not ordered, it is necessary to accurately capture appearance timings of the QRS complexes in 15 seconds to multiple minutes.

However, artifacts such as myoelectric noise may be mixed in the electrocardiogram. When detection of the QRS complexes is not accurate due to the mixing of noise into the electrocardiogram, following problems occur.

When noise is erroneously recognized as the QRS complex, the number of QRS complexes is measured more than actual, and the RR interval is measured smaller than actual. Accordingly, the heart rate is measured higher than actual. As a result, the heart rate displayed on the patient monitor does not decrease even when the heart rate actually decreases, and increases even when the heart rate actually does not increase. Accordingly, a false negative in a heart rate lower limit alarm and a false positive in a heart rate upper limit alarm may be generated.

When the QRS complex is erroneously recognized as noise, the number of QRS complexes is measured smaller than actual, and the RR interval is measured larger than actual. Accordingly, the heart rate is measured lower than actual. As a result, the heart rate displayed on the patient monitor does not increase even when the heart rate actually increases, and decreases even when the heart rate actually does not decrease. Accordingly, a false positive in the heart rate lower limit alarm and a false negative in the heart rate upper limit alarm may be generated.

When a signal quality of the electrocardiogram is determined to be low due to the mixing of noise, a method of regarding the electrocardiogram as not analyzable and displaying a heart rate measured in an immediate past without calculating the heart rate may be adopted. In this case, analysis for atrial fibrillation detection or the like is interrupted since the electrocardiogram is regarded as not analyzable, and as a result, detection of the atrial fibrillation or the like is interrupted, which is not preferable.

JPH11-47107A discloses a following related art. An electrocardiogram and pulse waves are measured, and QRS complexes of the electrocardiogram are detected to determine whether the QRS complexes are caused by premature ventricular contraction. Then, premature atrial contraction is detected by determining whether amplitudes of pulse waves corresponding to the QRS complexes determined not to be caused by premature ventricular contraction are smaller than a preset value.

However, the related art described above cannot cope with a high detection accuracy of the QRS complexes in the electrocardiogram.

### SUMMARY

The presently disclosed subject matter is made to solve the above-described problem. That is, an object of the presently disclosed subject matter is to provide a physiological information measurement system and a physiological information measurement method that can achieve a high detection accuracy of QRS complexes in an electrocardiogram.

The above object of the presently disclosed subject matter is solved by following configurations.
(1) A physiological information measurement system includes: a detector configured to detect a candidate QRS complex from an electrocardiogram of a subject; and an estimator configured to estimate whether the candidate QRS complex is a QRS complex based on the candidate QRS complex and information on a cardiac function of the subject.
(2) A physiological information measurement method includes: detecting a candidate QRS complex from an electrocardiogram of a subject; and estimating whether the candidate QRS complex is a QRS complex based on the candidate QRS complex and information on a cardiac function of the subject.

A candidate QRS complex is detected from an electrocardiogram of a subject, and is estimated to be a QRS complex or not based on the candidate QRS complex and information on a cardiac function of the subject. Accordingly, a high detection accuracy of the QRS wave in the electrocardiogram can be achieved.

### BRIEF DESCRIPTION OF DRAWINGS

FIG. 1 is a schematic configuration diagram of a physiological information measurement system.

FIG. 2 is a block diagram of a hardware configuration of a bedside monitor.

FIG. 3 is a block diagram illustrating functions of a controller.

FIG. 4 illustrates a general method for classifying a candidate QRS complex into a QRS complex or noise.

FIG. 5 illustrates an example of a method for determining presence and absence of a pulse wave corresponding to a candidate QRS complex.

FIG. 6 illustrates a comparative example of estimating a QRS complex based on a candidate QRS complex.

FIG. 7 illustrates another comparative example of estimating a QRS complex based on a candidate QRS complex.

FIG. 8 illustrates an example of estimating a QRS complex based on a candidate QRS complex.

FIG. 9 is a block diagram of a hardware configuration of a central monitor 200.

FIG. 10 is a flowchart illustrating operation of a bedside monitor 100.

FIG. 11 is a subroutine flowchart of step S103 of the flowchart in FIG. 10.

FIG. 12 is a subroutine flowchart of step S103 of the flowchart in FIG. 10.

FIG. 13 is a subroutine flowchart of step S103 of the flowchart in FIG. 10.

FIG. 14 illustrates a graph for estimating whether a candidate QRS complex is a QRS complex or noise based on a first probability and a second probability.

### DESCRIPTION OF EMBODIMENTS

Hereinafter, a physiological information measurement system and a physiological information measurement method according to embodiments of the presently disclosed subject matter will be described in detail with reference to the drawings. In the drawings, the same elements are denoted by the same reference numerals, and redundant description is omitted.

### First Embodiment

### Physiological Information Measurement System 10

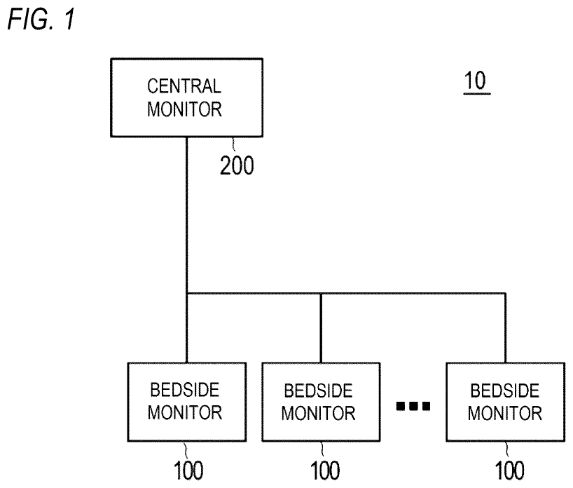

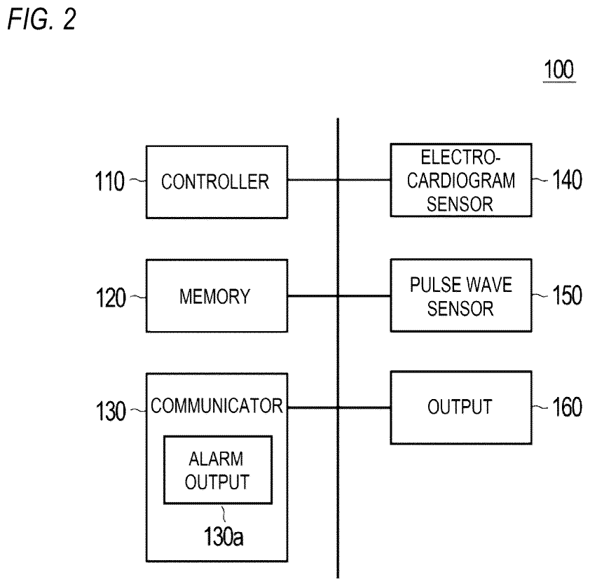

FIG. 1 is a schematic configuration diagram of a physiological information measurement system 10. The physiological information measurement system 10 can include bedside monitors 100 and a central monitor 200. One or more bedside monitors 100 may be provided. The bedside monitor 100 can include an electrocardiogram sensor 140 and a pulse wave sensor 150 (see FIG. 2). Alternatively, the electrocardiogram sensor 140 and the pulse wave sensor 150 may be devices separate from the bedside monitor 100. For example, the electrocardiogram sensor 140 and the pulse wave sensor 150 may be transmitters that can communicate with the bedside monitor 100 wirelessly, such as an electrocardiogram measurement device and a non-invasive blood pressure monitor. The physiological information measurement system 10 may include one bedside monitor 100.

The central monitor 200 and the bedside monitor 100 are communicably connected via a wired or wireless network. The network is, for example, a local area network (LAN) or a wide area network (WAN). As a communication standard of the network, for example, Ethernet (registered trademark), Wi-Fi (registered trademark), Bluetooth (registered trademark), or 5G may be used.

### Bedside Monitor 100

FIG. 2 is a block diagram of a hardware configuration of the bedside monitor 100. The bedside monitor 100 can include a controller 110, a memory 120, a communicator 130, the electrocardiogram sensor 140, the pulse wave sensor 150, and an output 160. These components are connected via a bus. The bedside monitor 100 is provided, for example, for each bed of a patient who is a measurement subject of physiological information, or for each room of patients.

The controller 110 can include, for example, a central processing unit (CPU) and a random access memory (RAM), controls the components of the bedside monitor 100, and performs various processes. Functions of the controller 110 will be described in detail later.

The memory 120 is, for example, a solid-state drive (SSD), and stores various data and various programs.

The communicator 130 is an interface for communicably connecting the bedside monitor 100 and other devices such as the central monitor 200. The communicator 130 may include, for example, an input terminal, an antenna, and a front-end circuit. The communicator 130 can include an alarm output 130a. The alarm output 130a transmits (outputs) an alarm to the central monitor 200. Specifically, the alarm output 130a transmits, for example, an alarm signal for notification of a heart rate abnormality to the central monitor 200.

The electrocardiogram sensor 140 can include, for example, a device, an element, and an electrocardiogram measurement electrode for detecting an electrocardiogram, and detects an electrocardiogram.

The pulse wave sensor 150 is, for example, a SpO2 probe, an invasive blood pressure monitor (including a catheter and the like) or a non-invasive blood pressure monitor (including a cuff and the like), and detects a pulse wave. The pulse wave sensor 150 measures an SpO2 waveform or a blood pressure waveform, and detects a pulse wave based on the measured waveform. The pulse wave sensor 150 may be replaced with a cardiac sound sensor.

The functions of the controller 110 will be described.

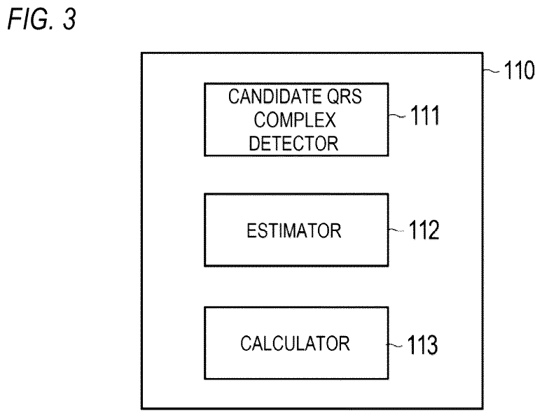

FIG. 3 is a block diagram illustrating the functions of the controller 110. The controller 110 functions as a candidate QRS complex detector 111, an estimator 112, and a calculator 113 by executing programs. The candidate QRS complex detector 111 constitutes a detector.

The candidate QRS complex detector 111 obtains, from the electrocardiogram sensor 140, an electrocardiogram of a subject who is a person to be measured of a heart rate, and detects a candidate QRS complex from the electrocardiogram. The candidate QRS complex is detected from the electrocardiogram detected by the electrocardiogram sensor 140, and is a candidate of a QRS complex. The candidate QRS complex is detected by a freely selected known method as long as it is a method for detection based on only information on an electrocardiogram. For example, the candidate QRS complex is detected by filtering the electrocardiogram with a band-pass filter and then detecting a waveform peak. The electrocardiogram detected by the electrocardiogram sensor 140 may contain noise such as myoelectric noise. For this reason, noise (noise waveform) may be detected as a candidate QRS complex. A QRS complex on which noise is superimposed may not be detected as a candidate QRS complex due to noise superimposition.

The estimator 112 estimates whether a candidate QRS complex is a QRS complex based on the candidate QRS complex and information on a cardiac function of the subject. The information on the cardiac function of the subject can include a pulse wave, a cardiac sound, and the like. Hereinafter, to simplify the description, a case where the information on the cardiac function of the subject is a pulse wave will be described as an example.

The estimator 112 estimates whether a candidate QRS complex is a QRS complex by classifying the candidate QRS complex into the QRS complex or noise based on presence and absence of a pulse wave corresponding to the candidate QRS complex (information on the cardiac function).

As a general method for classifying the candidate QRS complex into the QRS complex or noise, a method of determining whether the candidate QRS complex has noise features is considered. Following items are considered as the noise features.
The number of peaks in a vicinity of the candidate QRS complex in the electrocardiogram is large (equal to or greater than a prescribed threshold) or small (less than a prescribed threshold).
The number of times the waveform intersects a baseline in the vicinity of the candidate QRS complex in the electrocardiogram is large (equal to or greater than a prescribed threshold) or small (less than a prescribed threshold).
A waveform variation (standard deviation) in the vicinity of the candidate QRS complex in the electrocardiogram is large (equal to or greater than a prescribed threshold) or small (less than a prescribed threshold).
A similarity between the candidate QRS complex and a previously stored QRS complex template is low (equal to or less than a prescribed threshold).

The prescribed thresholds described above may be set to be appropriate values from a viewpoint of a detection accuracy of the QRS complex.

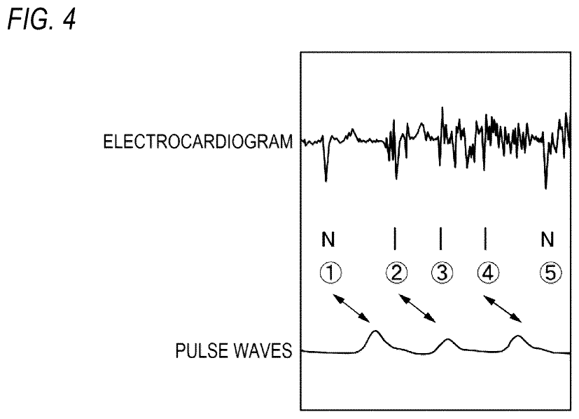

FIG. 4 illustrates a general method for classifying a candidate QRS complex into a QRS complex or noise.

FIG. 4 illustrates an electrocardiogram, detection positions of candidate QRS complexes in the electrocardiogram and classification results of the candidate QRS complexes, and pulse waves. The detection positions of the candidate QRS complexes in the electrocardiogram and the classification results of the candidate QRS complexes in the electrocardiogram are indicated by beat labels of “N” or “|”. The beat labels can include “N” indicating a QRS complex and “|” indicating noise (not QRS complex). In FIG. 4, the beat labels “|” to which circled characters “2”, “3”, and “4” are attached has the noise features described above, and can thus be classified into noise by a general method for classifying the candidate QRS complex into the QRS complex or noise. However, among the candidate QRS complexes of “2”, “3”, and “4” displayed with the beat label “|”, the candidate QRS complexes of “2” and “4” have corresponding pulse waves. Here, “corresponding” refers to arising from the same heartbeat. Accordingly, the candidate QRS complexes of “2” and “4” displayed with the beat label “|” are determined as not actually noise but QRS complexes, and are erroneously classified into noise.

The estimator 112 classifies a candidate QRS complex based on presence and absence of a pulse wave corresponding to the candidate QRS complex. Accordingly, accuracy of classifying the candidate QRS complex into the QRS complex or noise can be improved.

The estimator 112 may determine the presence and absence of the pulse wave corresponding to the candidate QRS complex based on whether a pulse wave is detected within a prescribed time delay range after the candidate QRS complex is detected. That is, the estimator 112 determines the pulse wave detected within the prescribed time delay range after the candidate QRS complex is detected as the pulse wave corresponding to the candidate QRS complex. The prescribed time delay range may be set to be an appropriate range by tests from a viewpoint of a detection accuracy of the QRS complex.

The estimator 112 may adopt a pulse wave satisfying at least one of following conditions as a pulse wave used for determining the presence and absence of the pulse wave corresponding to the candidate QRS complex.
An amplitude and a slope of a waveform are within prescribed ranges from average values of past pulse waves.
An interval of the waveforms is constant (within a prescribed range).
A similarity between the waveform and a template waveform of the pulse wave or an adjacent waveform in time series is greater than or equal to a prescribed value.

The prescribed ranges and the prescribed values may be set to be appropriate values from the viewpoint of the detection accuracy of the QRS complex.

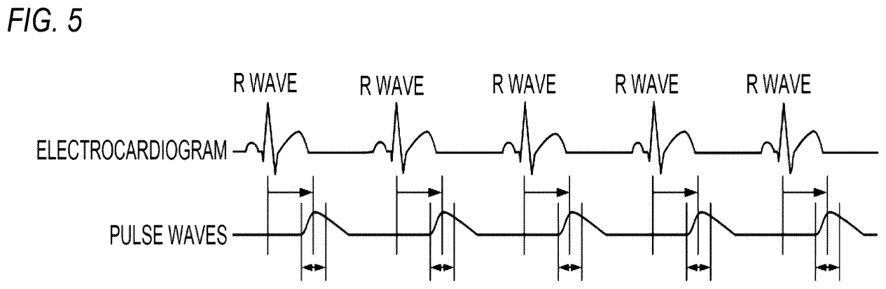

FIG. 5 illustrates an example of a method for determining presence and absence of a pulse wave corresponding to a candidate QRS complex. FIG. 5 illustrates an electrocardiogram and pulse waves with a common timeline.

The pulse wave transferred via blood in blood vessels is detected later than a QRS complex. The pulse wave (more specifically, peak of the pulse wave) is detected with a time delay (length of a unidirectional arrow in FIG. 5) relative to the QRS complex (more specifically, R wave).

A prescribed time delay range may be a range having a prescribed width (length of a bidirectional arrow in FIG. 5) centered on an average time delay of past beats of the same subject (patient or the like) during heart rate measurement. For example, the prescribed time delay range is a range having a width of 40 ms before and after the average time delay of past beats of the same subject during the heart rate measurement. The prescribed time delay range may also be a range having a width of 10 ms to 100 ms before and after the average time delay of past beats of the same subject.

When the information on the cardiac function of the subject is a cardiac sound, a sound I and a sound II are preferably used as cardiac sounds for determining presence and absence of a cardiac sound corresponding to a candidate QRS complex. In a phonocardiogram, a valve sound generated during cardiac contraction is measured. The sound I is a sound associated with mitral and tricuspid valve closure. The sound II is a sound associated with aorta and pulmonary valve closure. The estimator 112 may determine the presence and absence of the cardiac sound corresponding to the candidate QRS complex based on whether the sound I or the sound II is detected within a prescribed time delay range after the candidate QRS complex is detected. When reliability is emphasized, detection of both the sound I and the sound II may be required.

### Comparative Example 1

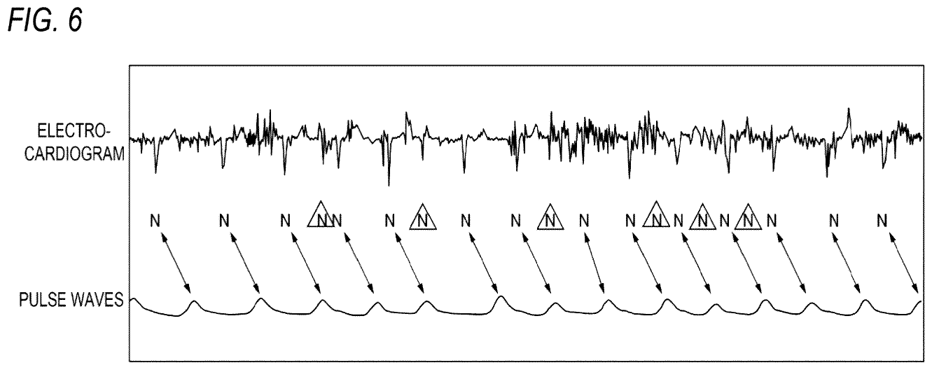

FIG. 6 illustrates a comparative example of estimating a QRS complex based on a candidate QRS complex. FIG. 6 illustrates an example when all candidate QRS complexes are estimated as QRS complexes as a result of classifying the candidate QRS complexes by the above-described general method. Detection positions of the candidate QRS complexes in an electrocardiogram and classification results of the candidate QRS complexes are indicated by the beat label of “N”. Although pulse waves are not used in the estimation of the QRS complexes in the present comparative example, the pulse waves are also illustrated in FIG. 6 for convenience of description.

Noise such as myoelectric noise is mixed in the electrocardiogram. For this reason, noise that is not a QRS complex may be detected as a candidate QRS complex. In the present comparative example, as a result of estimating all the candidate QRS complexes as QRS complexes, noise that is not a QRS complex is erroneously estimated as a QRS complex. Among the beat labels, beat labels that are erroneously estimated as QRS complexes despite being noise are surrounded by triangles. In FIG. 6, correspondence relationships between the candidate QRS complexes and the pulse waves are indicated by bidirectional arrows, and the beat labels erroneously estimated as QRS complexes despite being noise are beat labels estimated as QRS complexes despite absence of corresponding pulse waves.

When noise is erroneously estimated as QRS complexes as in the present comparative example, a heart rate calculated based on the QRS complexes is calculated to be higher than actual, and a measurement accuracy of the heart rate decreases.

### Comparative Example 2

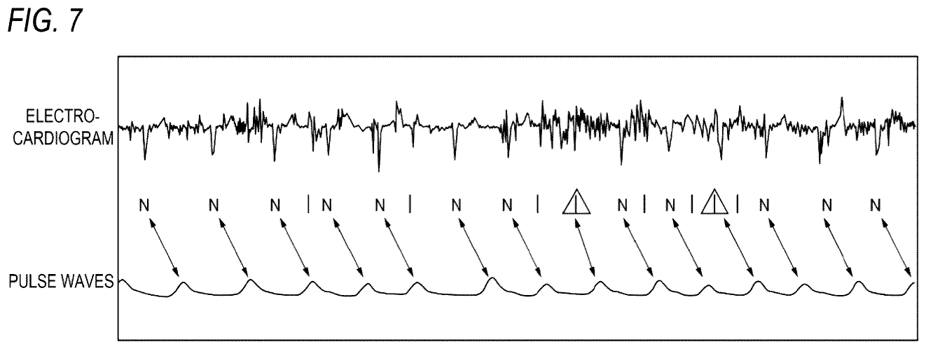

FIG. 7 illustrates another comparative example of estimating a QRS complex based on a candidate QRS complex. FIG. 7 illustrates a case in which, as a result of classifying candidate QRS complexes by the above-described general method, a part of candidate QRS complexes are estimated as noise despite being actually QRS complexes. Although pulse waves are not used in the estimation of the QRS complexes in the present comparative example, the pulse waves are also illustrated in FIG. 7 for convenience of description.

Noise such as myoelectric noise is mixed in the electrocardiogram. For this reason, the candidate QRS complexes may be estimated as noise despite being actually QRS complexes due to an influence of noise. In the present comparative example, a part of candidate QRS complexes are estimated as noise despite being actually QRS complexes. Among the beat labels, beat labels that are erroneously estimated as noise despite being QRS complexes are surrounded by triangles. In FIG. 7, correspondence relationships between the candidate QRS complexes and the pulse waves are indicated by bidirectional arrows, and the beat labels erroneously estimated as noise despite being QRS complexes are beat labels estimated as noise despite absence of corresponding pulse waves.

When the QRS complexes are erroneously estimated as noise as in the present comparative example, a heart rate calculated based on the QRS complexes is calculated to be lower than actual, and a measurement accuracy of the heart rate decreases.

### Example

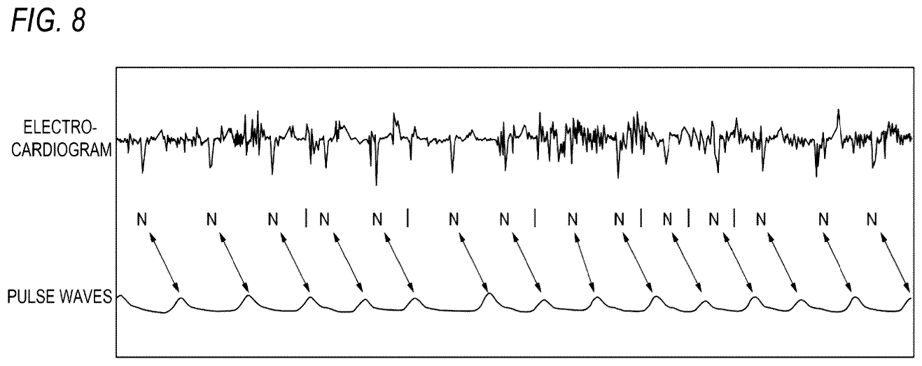

FIG. 8 illustrates an example of estimating a QRS complex based on a candidate QRS complex.

As illustrated in FIG. 8, in the example, the beat label of “N” is displayed as classification results of candidate QRS complexes having corresponding pulse waves, and the beat label of “|” is displayed as classification results of candidate QRS complexes having no corresponding pulse waves. That is, the candidate QRS complexes having corresponding pulse waves are estimated as QRS complexes, and the candidate QRS complexes having no corresponding pulse waves are estimated as noise. In FIG. 8, correspondence relationships between candidate QRS complexes and pulse waves are indicated by bidirectional arrows.

By classifying the candidate QRS complexes based on presence and absence of pulse waves corresponding to the candidate QRS complexes as in the example, an estimation accuracy of the QRS complexes can be prevented from decreasing due to noise.

The calculator 113 calculates a heart rate based on the estimated QRS complexes (candidate QRS complexes classified into QRS complexes). Specifically, the controller 110 calculates an RR interval from the estimated QRS complexes and calculates the heart rate from the RR interval. The controller 110 determines whether the heart rate is an abnormal value based on whether the heart rate exceeds a prescribed normal range. The prescribed normal range is set to be, for example, 60 to 100. When determining that the heart rate is an abnormal value, the controller 110 issues a heart rate alarm to the output 160. When determining that the heart rate is an abnormal value, the controller 110 may issue an alarm by transmitting a heart rate alarm signal to the central monitor 200 by the alarm output 130a together with an electrocardiogram, pulse waves, and information specifying a subject.

The output 160 may include a display and a speaker. The output 160 may give a notification by displaying the heart rate alarm on the display in highlighted characters or the like. The output 160 may give a notification of the heart rate alarm by outputting the heart rate alarm from the speaker in audio, warning sound, and the like.

### Central Monitor 200

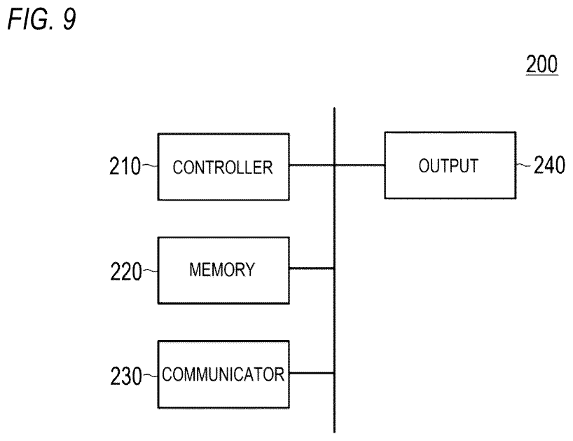

FIG. 9 is a block diagram of a hardware configuration of the central monitor 200. The central monitor 200 can include a controller 210, a memory 220, a communicator 230, and an output 240. These components are connected via a bus. Among these components of the central monitor 200, basic configurations of components corresponding to components of the bedside monitor 100 are the same as or similar to basic configurations of the bedside monitor 100, and thus redundant description is omitted.

The controller 210 receives, from each bedside monitor 100, an electrocardiogram, pulse waves, a heart rate alarm signal, and information specifying a subject, by the communicator 230.

The controller 210 may give a notification of the heart rate alarm by the output 240 by displaying the electrocardiogram, the pulse waves, and the heart rate alarm signal on the display in highlighted characters or the like for each subject specified by the information specifying the subject. The output 160 may give a notification of the heart rate alarm by outputting the heart rate alarm from the speaker in audio, warning sound, and the like.

Operation of the physiological information measurement system 10 will be described.

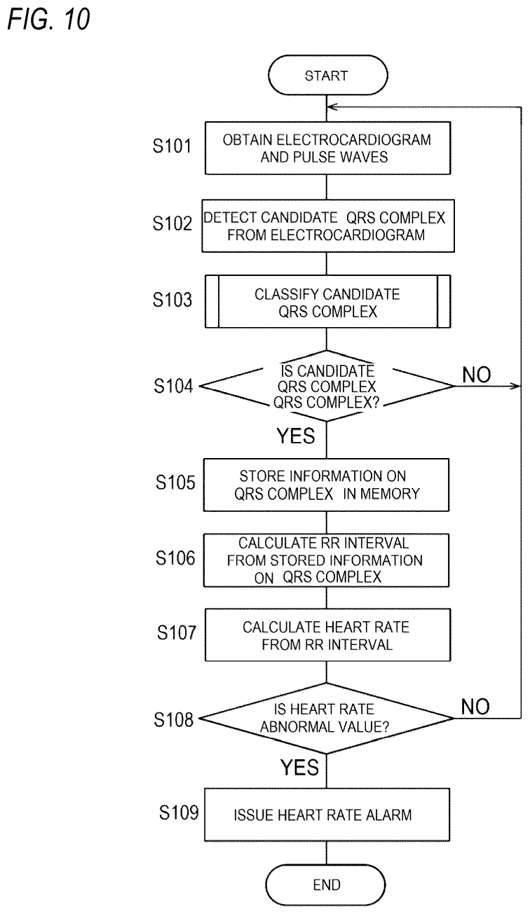

FIG. 10 is a flowchart illustrating operation of the bedside monitor 100. The flowchart may be executed by the controller 110 of the bedside monitor 100 according to a program.

The controller 110 obtains an electrocardiogram and pulse waves respectively from the electrocardiogram sensor 140 and the pulse wave sensor 150 (S101).

The controller 110 detects a candidate QRS complex from the electrocardiogram (S102).

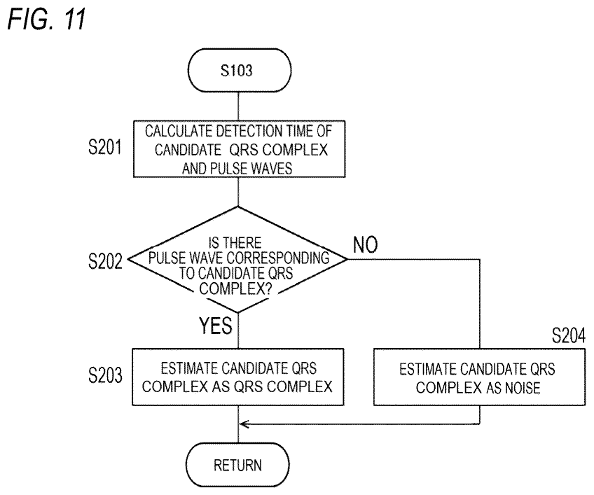

The controller 110 classifies the candidate QRS complex into a QRS complex or noise (S103). Details of this step will be described later with reference to a subroutine flowchart of FIG. 11.

When the candidate QRS complex is not classified into the QRS complex but is classified into noise (S104: NO), the controller 110 returns to step S101 and continues the process.

When the candidate QRS complex is classified into the QRS complex (S104: YES), the controller 110 stores information on the QRS complex in the memory (S105).

The controller 110 calculates an RR interval from the stored information on the QRS complex (S106), and calculates a heart rate from the RR interval (S107).

The controller 110 determines whether the heart rate is an abnormal value (S108). When determining that the heart rate is not an abnormal value (S108: NO), the controller 110 returns to step S101 and continues the process.

When determining that the heart rate is an abnormal value (S108: YES), the controller 110 issues a heart rate alarm to the output 160 or the like (S109).

FIG. 11 is a subroutine flowchart of step S103 of the flowchart of FIG. 10.

The controller 110 calculates detection time (generation time) of the candidate QRS complex and the pulse waves (S201).

Based on the detection time of the candidate QRS complex and the pulse waves, the controller 110 determines presence and absence of a pulse wave corresponding to the candidate QRS complex based on whether a pulse wave is detected within a prescribed time delay range after the candidate QRS complex is detected (S202). When determining no pulse wave corresponding to the candidate QRS complex (S202: NO), the controller 110 estimates the candidate QRS complex as noise (S204).

When determining a pulse wave corresponding to the candidate QRS complex (S202: YES), the controller 110 estimates the candidate QRS complex as the QRS complex (S203).

### Modification 1

A modification of the operation of the physiological information measurement system 10 will be described. In the present modification, a candidate QRS complex is classified into a QRS complex or noise by the above-described general method based on the candidate QRS complex alone, and then the QRS candidate classified into noise is reclassified into the QRS complex or noise depending on presence and absence of a corresponding pulse wave.

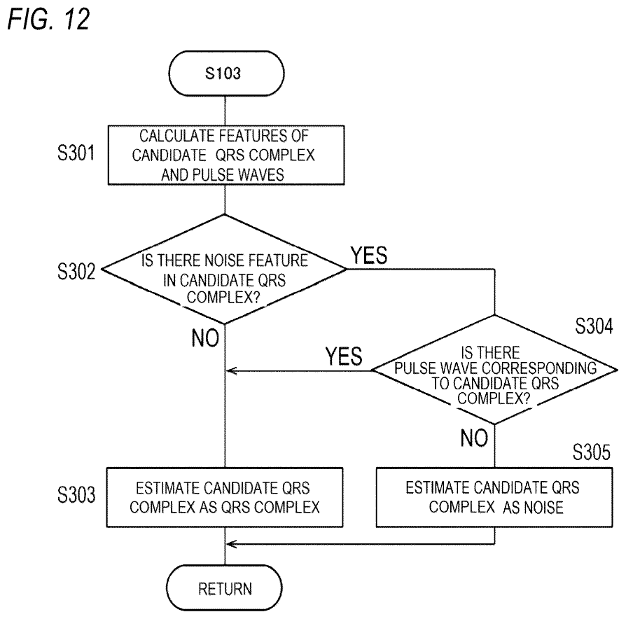

FIG. 12 is a subroutine flowchart of step S103 of the flowchart of FIG. 10.

The controller 110 calculates features of the candidate QRS complex and the pulse waves (S301). The feature of the candidate QRS complex can include detection time (generation time) of the candidate QRS complex and a feature for determining presence and absence of a noise feature. Specifically, the feature for determining presence and absence of the noise feature can include a general method for classifying the candidate QRS complex into the QRS complex or noise. The feature of the pulse waves can include detection time (generation time) of the pulse waves.

The controller 110 determines whether there is a noise feature in the candidate QRS complex based on the feature of the candidate QRS complex (S302). When determining no noise feature in the candidate QRS complex (S302: NO), the controller 110 estimates the candidate QRS complex as the QRS complex.

When determining a noise feature in the candidate QRS complex (S302: YES), the controller 110 determines presence and absence of a pulse wave corresponding to the candidate QRS complex based on the detection time of the candidate QRS complex and the detection time of the pulse waves (S304).

When determining a pulse wave corresponding to the candidate QRS complex (S304: YES), the controller 110 estimates the candidate QRS complex as the QRS complex (S303). When determining no pulse wave corresponding to the candidate QRS complex (S204: NO), the controller 110 estimates the candidate QRS complex as noise (S305).

### Modification 2

Another modification of the operation of the physiological information measurement system 10 will be described. In the present modification, a candidate QRS complex is classified into a QRS complex or noise by the above-described general method based on the candidate QRS complex alone, and then the QRS candidate classified into the QRS complex is reclassified into the QRS complex or noise depending on presence and absence of a corresponding pulse wave.

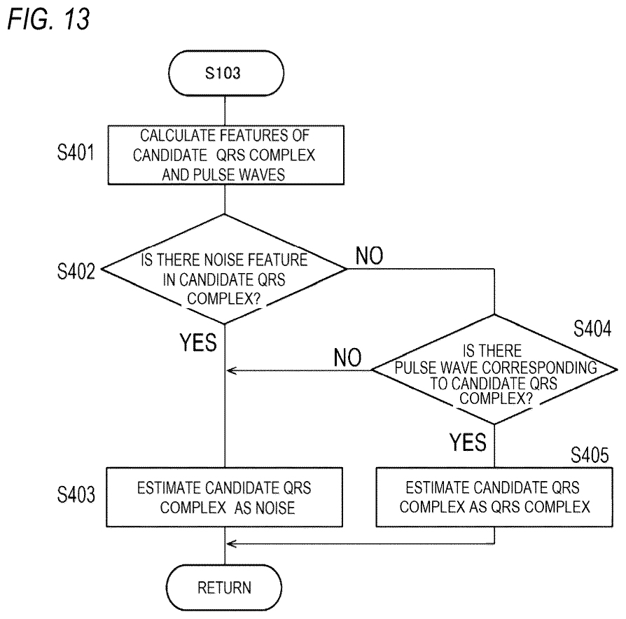

FIG. 13 is a subroutine flowchart of step S103 of the flowchart of FIG. 10.

The controller 110 calculates features of the candidate QRS complex and the pulse waves (S401). The feature of the candidate QRS complex and the feature of the pulse waves are the same as or similar to those in Modification 1 described above.

The controller 110 determines whether there is a noise feature in the candidate QRS complex based on the feature of the candidate QRS complex (S402). When determining a noise feature in the candidate QRS complex (S402: YES), the controller 110 estimates the candidate QRS complex as noise.

When determining no noise feature in the candidate QRS complex (S402: NO), the controller 110 determines presence and absence of a pulse wave corresponding to the candidate QRS complex based on the detection time of the candidate QRS complex and the detection time of the pulse waves (S404).

When determining a pulse wave corresponding to the candidate QRS complex (S404: YES), the controller 110 estimates the candidate QRS complex as the QRS complex (S405). When determining no pulse wave corresponding to the candidate QRS complex (S404: NO), the controller 110 estimates the candidate QRS complex as noise (S403).

### Second Embodiment

A second embodiment will be described. The present embodiment is different from the first embodiment in following aspects. In the first embodiment, a candidate QRS complex is estimated to be a QRS complex or not based on presence and absence of a pulse wave corresponding to the candidate QRS complex. On the other hand, in the present embodiment, the candidate QRS complex is estimated to be a QRS complex or not based on a probability of the candidate QRS complex being a QRS complex based on the candidate QRS complex and a probability of a pulse wave corresponding to the candidate QRS complex. The present embodiment is the same as or similar to the first embodiment in other respects, and thus redundant description is omitted or simplified.

The estimator 112 calculates the probability of the candidate QRS complex being a QRS complex (hereinafter also referred to as a “first probability”) based on the candidate QRS complex. The first probability may be calculated, for example, by setting an initial value to be 0.5, multiplying the number of conditions among following conditions that are satisfied by the candidate QRS complex by 0.1, and adding the resultant number to the initial value.
The number of peaks in the candidate QRS complex is less than a prescribed threshold.
The number of times that the candidate QRS complex intersects a baseline is less than a prescribed threshold.
A waveform variation (standard deviation) of the candidate QRS complex is less than a prescribed threshold.
A degree of similarity between the candidate QRS complex and a QRS template is less than a prescribed threshold.

The prescribed thresholds described above may be set to be appropriate values from a viewpoint of a detection accuracy of the QRS complex. Conditions for calculating the first probability may be a part of the above conditions, and other conditions may be added.

The estimator 112 calculates the probability of a pulse wave corresponding to the candidate QRS complex (hereinafter also referred to as a “second probability”) based on the candidate QRS complex and pulse waves. The second probability may be calculated, for example, by setting an initial value to be 0.5, multiplying the number of conditions among following conditions that are satisfied by the candidate QRS complex or the pulse waves by 0.1, and adding the resultant number to the initial value.
Time from detection time (generation time) of the QRS candidate to detection time (generation time) of a pulse wave is within a prescribed range centered on a past average value.
An amplitude of the pulse wave is within a prescribed range centered on a past average value.
A slope (differential value) of the pulse wave is within a prescribed range centered on a past average value.
A degree of similarity between the pulse wave and a pulse wave template is greater than or equal to a prescribed threshold.

The prescribed ranges and the prescribed thresholds may be set to be appropriate values from a viewpoint of a detection accuracy of the QRS complex. Conditions for calculating the second probability may be a part of the above conditions, and other conditions may be added.

The estimator 112 estimates whether the candidate QRS complex is a QRS complex or noise based on the first probability and the second probability.

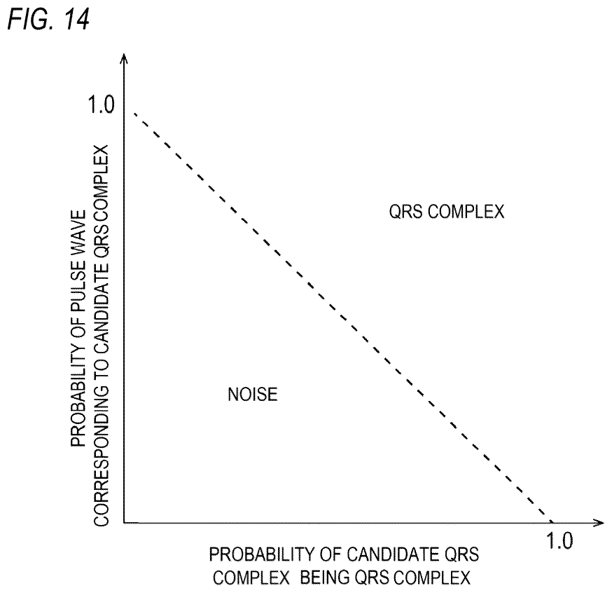

FIG. 14 illustrates a graph for estimating whether a candidate QRS complex is a QRS complex or noise based on the first probability and the second probability.

Whether the candidate QRS complex is a QRS complex or noise is estimated as follows using, for example, the graph of FIG. 14.

In the graph of FIG. 14, a horizontal axis (x coordinate) is the first probability, and a vertical axis (y coordinate) is the second probability. In the graph, a straight line connecting a point with the first probability being 1.0 on the horizontal axis and a point with the second probability being 1.0 on the vertical axis is indicated by a broken line. A region on an origin side of the graph relative to the threshold straight line is a noise region, and a region on a side opposite to a graph origin relative to the threshold straight line is a QRS complex region. The candidate QRS complex is estimated as noise or a QRS complex based on whether coordinates (x, y)=(first probability, second probability) of a combination of the first probability and the second probability belong to the noise region or QRS complex region.

The embodiments achieve following effects.

A candidate QRS complex is detected from an electrocardiogram of a subject, and is estimated to be a QRS complex or not based on the candidate QRS complex and information on a cardiac function of the subject. Accordingly, a high detection accuracy of the QRS wave in the electrocardiogram can be achieved.

Further, a heart rate is calculated based on the waveform estimated as QRS. This can improve a measurement accuracy of the heart rate based on the electrocardiogram.

The information on the cardiac function is referred to as a pulse wave or a cardiac sound. Accordingly, a high detection accuracy of the QRS wave in the electrocardiogram can be achieved simply and effectively.

Further, a candidate QRS complex is estimated to be a QRS complex or not based on presence and absence of information on the cardiac function corresponding to the candidate QRS complex. Accordingly, a high detection accuracy of the QRS wave in the electrocardiogram can be achieved effectively.

Further, the presence and absence of the information on the cardiac function corresponding to the candidate QRS complex is determined based on whether information on the cardiac function is detected within a prescribed time delay range after the candidate QRS complex is detected. Accordingly, the high detection accuracy of the QRS wave in the electrocardiogram can be further improved.

Information on the cardiac function used for determining the presence and absence of the information on the cardiac function corresponding to the candidate QRS complex satisfies at least one of following conditions: the information is a pulse wave or a cardiac sound, an amplitude and a slope of a waveform are within prescribed ranges from past average values, an interval between the waveforms is constant, and a degree of similarity between the waveform and a template waveform or an adjacent waveform in time series is smaller than or equal to a prescribed value. Accordingly, the high detection accuracy of the QRS wave in the electrocardiogram can be further improved.

Further, a heart rate is calculated by calculating an RR interval of the waveform estimated as QRS. Accordingly, the heart rate based on the electrocardiogram can be measured more easily.

Further, the candidate QRS complex is estimated to be a QRS complex or not based on a probability of the candidate QRS complex being a QRS complex based on the candidate QRS complex and a probability of a pulse wave corresponding to the candidate QRS complex. Accordingly, the high detection accuracy of the QRS wave in the electrocardiogram can be further improved.

Although the embodiments of the presently disclosed subject matter are described in detail above, the presently disclosed subject matter is not limited to the above-described embodiments.

For example, a part or all of functions and configurations of the bedside monitor 100 may be contained in functions and configurations of the central monitor 200. In this case, the bedside monitor 100 may be replaced with a transmitter, and an electrocardiogram and pulse waves may be transmitted from each transmitter to the central monitor 200.

For example, a part or all of functions implemented by programs in the above-described embodiments may be implemented by hardware such as a circuit.

In the flowcharts described above, a part of steps may be omitted, and other steps may be added. A part of the steps may be executed at the same time, or one step may be divided into a plurality of steps and executed.

1. A physiological information measurement system comprising:
a detector configured to detect a candidate QRS complex from an electrocardiogram of a subject; and
an estimator configured to estimate whether the candidate QRS complex is a QRS complex based on the candidate QRS complex and information on a cardiac function of the subject.

2. The physiological information measurement system according to claim 1, further comprising:
a calculator configured to calculate a heart rate based on the waveform estimated as QRS by the estimator.

3. The physiological information measurement system according to claim 1, wherein
the information on the cardiac function is a pulse wave or a cardiac sound.

4. The physiological information measurement system according to claim 1, wherein
the estimator estimates whether the candidate QRS complex is the QRS complex based on presence and absence of information on the cardiac function corresponding to the candidate QRS complex.

5. The physiological information measurement system according to claim 4, wherein
the estimator determines the presence and absence of the information on the cardiac function corresponding to the candidate QRS complex based on whether information on the cardiac function is detected within a prescribed time delay range after the candidate QRS complex is detected.

6. The physiological information measurement system according to claim 4, wherein
the estimator uses information on the cardiac function, which is used for determining the presence and absence of the information on the cardiac function corresponding to the candidate QRS complex, that satisfies at least one of following conditions:
the information is a pulse wave or a cardiac sound;
an amplitude and a slope of a waveform are within prescribed ranges from past average values;
an interval of the waveforms is constant; and
a degree of similarity between the waveform and a template waveform or an adjacent waveform in time series is greater than or equal to a prescribed value.

7. The physiological information measurement system according to claim 2, wherein
the calculator calculates a heart rate by calculating an RR interval of the waveform estimated as QRS by the estimator.

8. The physiological information measurement system according to claim 1, wherein
the estimator estimates whether the candidate QRS complex is the QRS complex based on a probability of the candidate QRS complex being the QRS complex based on the candidate QRS complex and a probability of a pulse wave corresponding to the candidate QRS complex.

9. A physiological information measurement method comprising:
detecting a candidate QRS complex from an electrocardiogram of a subject; and
estimating whether the candidate QRS complex is a QRS complex based on the candidate QRS complex and information on a cardiac function of the subject.

10. The physiological information measurement method according to claim 9, further comprising:
calculating a heart rate based on the waveform estimated as QRS in the estimating.

11. The physiological information measurement method according to claim 9, wherein
the information on the cardiac function is a pulse wave or a cardiac sound.

12. The physiological information measurement method according to claim 9, wherein
in the estimating, whether the candidate QRS complex is the QRS complex is estimated based on presence and absence of information on the cardiac function corresponding to the candidate QRS complex.

13. The physiological information measurement method according to claim 12, wherein
in the estimating, the presence and absence of the information on the cardiac function corresponding to the candidate QRS complex is determined based on whether information on the cardiac function is detected within a prescribed time delay range after the candidate QRS complex is detected.

14. The physiological information measurement method according to claim 12, wherein
in the estimating, information on the cardiac function used for determining the presence and absence of the information on the cardiac function corresponding to the candidate QRS complex satisfies at least one of following conditions:
the information is a pulse wave or a cardiac sound;
an amplitude and a slope of a waveform are within prescribed ranges from past average values;
an interval of the waveforms is constant; and
a degree of similarity between the waveform and a template waveform or an adjacent waveform in time series is greater than or equal to a prescribed value.

15. The physiological information measurement method according to claim 10, wherein
in the calculating, a heart rate is calculated by calculating an RR interval of the waveform estimated as QRS in the estimating.

16. The physiological information measurement method according to claim 9, wherein
in the estimating, whether the candidate QRS complex is the QRS complex is estimated based on a probability of the candidate QRS complex being the QRS complex based on the candidate QRS complex and a probability of a pulse wave corresponding to the candidate QRS complex.
